(19) United States
(12) Patent Application Publication
(10) Pub. No.: US 2025/0338114 A1
(43) Pub. Date: Oct. 30, 2025

(54) INFUSION PUMP SYSTEM, METHOD FOR SECURE DATA TRANSMISSION FOR INFUSION PUMP SYSTEM AND COMPUTER-READABLE STORAGE MEDIUM

(71) Applicant: B. Braun Melsungen AG, Melsungen (DE)

(72) Inventors: Dennis Obermann, Willingshausen (DE); Sebastian Moeller, Hessisch Lichtenau (DE)

(21) Appl. No.: 19/185,516

(22) Filed: Apr. 22, 2025

(30) Foreign Application Priority Data
Apr. 24, 2024 (EP) 24172092.9

(51) Int. Cl. H04W 12/037; A61M 5/142; G16H 40/67; H04L 9/32; H04W 12/50

(52) U.S. Cl. CPC H04W 12/037; A61M 5/14212; G16H 40/67; H04L 9/3247; H04W 12/50; A61M 2205/3569

(57) ABSTRACT

An infusion pump system has a first infusion pump and a second infusion pump that are paired or pairable to one another for secure data transmission. At least one of the first and second infusion pumps receives data signed with a digital signature, and/or at least one of the first and second infusion pumps receives data encrypted with an encryption. A method can be used for secure data transmission for the infusion pump system. The secure data transmission method can be performed using a computer-readable storage medium having functions that cause the infusion pump system to perform the method.

### CROSS-REFERENCE TO RELATED APPLICATIONS

This application claims priority under 35 U.S.C. § 119 to European Application No. 24172092.9, filed on Apr. 24, 2024, the content of which is incorporated by reference herein in its entirety.

### FIELD

The disclosure relates to an infusion pump system, a method for secure data transmission for an infusion pump system, and a computer-readable storage medium.

### BACKGROUND

In automated infusion technology, infusion pumps are paired to each other to provide therapies together. One example of this is the so-called takeover mode. This supports the user during syringe changes on a syringe pump by automatically starting a second syringe pump as soon as the first syringe pump has run dry. For this purpose, information is exchanged between the syringe pump that is running empty and the syringe pump that will subsequently take over the treatment. These are necessary to synchronize infusion parameters or to initiate actions such as starting the receiving syringe pump.

With current Take-Over-Mode implementations and other implementations for data transmission between infusion pumps, this information is not transmitted securely. It is therefore possible that this information could be tapped and manipulated.

### SUMMARY

In view of the problem described above, it is therefore one task of the disclosure to ensure a secure data transfer in an infusion pump system with at least two infusion pumps, or at least to make the data transmission more secure.

Advantageous embodiments are described in the following description.

The disclosure-based infusion pump system has a first infusion pump and a second infusion pump that are/can be paired to one another for secure data transmission, wherein the infusion pump system is set up or configured so that at least one of the first and second infusion pumps receives data signed with a digital signature and/or at least one of the first and second infusion pumps receives data encrypted with an encryption.

Encrypted data or encrypted should therefore be understood to mean that the data is encrypted with an appropriate encryption. signed data or signed is therefore to be understood as meaning that the data is signed with a corresponding digital signature.

A pairing is to be understood as the creation of a possibility/line/connection with which/over which data can be transmitted securely, preferably to make it possible to carry out therapy from several infusion pumps securely at the same time and/or in succession. This means that paired infusion pumps can securely transmit data by apparently encrypting and/or digitally signing them, preferably in order to jointly carry out a therapy. Secure data transmission can be carried out, for example, via cable or wirelessly and/or via an internal hospital network, for example. Such a network can also provide already encrypted lines, e.g., point-to-point encryption, or the lines can be unencrypted.

The data includes, for example, status reports, messages, and instructions. An instruction can be, for example, to start the infusion pump, to take over the therapy, or a command to pair to at least one other infusion pump and/or to another infusion pump, or to request a pairing. A status message can, for example, be the amount of a drug that is available, a fault report or a status report, such as ready for use, active, on stand-by, or currently being serviced.

An infusion pump that sends instruction(s) and/or command(s) to another infusion pump can be designated as the master, especially in a take-over mode. Accordingly, the infusion pump receiving the instruction(s) can be designated as the slave. A master can, for example, carry out a therapy and instruct the slave to adopt this therapy.

It is to be understood that the data transmission is/can be designated as secure because, according to the disclosure, the data is/will be provided with a digital signature and/or is/will be encrypted.

It should be understood that a digital signature in cryptology is used by the sender of digitally signed data to clearly identify that the data originates from him. In other words, the digital signature is used so that a recipient of the data can use the digital, unique, and tamper-proof signature to check whether the data really originate from the sender or whether someone else sent the data and/or tapped and modified it before sending it to the recipient. A digital signature is therefore a kind of ID card. The data can, for example, be digitally signed/authenticated and checked/validated based on known digital signature methods, such as RSA, DAS, El-Gamal, or methods based on them, or other methods. Preferably, the digital signature is generated based on an asymmetric cryptosystem.

It should be understood that encrypted data in cryptography/cryptology is used to prevent unauthorized persons from decrypting and reading it. The encrypted data is preferably encrypted and decrypted based on asymmetric encryption methods or public-key encryption methods or asymmetric cryptosystems, such as RSA or methods based on it. However, the disclosure is not limited to this; data can also be encrypted using symmetric encryption methods.

It is to be understood that, according to the disclosures, a common method or several different methods or the same method can be used separately/repeatedly for encrypting and decrypting and/or digitally signing and verifying digital signatures.

It is further to be understood that data that is encrypted and/or digitally signed and that one of the two infusion pumps receives may originate from the other one of the two infusion pumps, or also from a further infusion pump or another device, such as a computer. In other words, the infusion pump system may also include other infusion pumps or other devices and is not limited to exactly two infusion pumps.

The term “receiving” may also include receiving, processing, evaluating, decrypting, or validation.

The advantages of the disclosure are that at least two infusion pumps can securely transmit data with each other and that at least one pump can securely receive data. This means that the two infusion pumps can work together safely, for example to administer a patient's therapy. In particular for a take-over mode, it is important that the data is transmitted reliably and securely so that there can be no pause during therapy. The patient is protected from the data that the infusion pumps receive/exchange being manipulated and/or originating from an unauthorized/third-party device. Furthermore, the secure data transmission in accordance with the disclosure is suitable for e.g. an in-house and/or wireless network, such as WLAN, so that it is not necessary for the infusion pumps to be connected by cable. This saves on components and eliminates the need to connect or disconnect infusion pumps by cable. A connection/pairing via a wireless network is more flexible. Furthermore, the secure data transmission between the infusion pumps is inexpensive, since only the communication between the infusion pumps or at least to one infusion pump/in the infusion pump system is secure and thus only the data received there is encrypted or digitally signed. It is therefore not necessary for all infusion pumps to transmit data securely at all times or for an entire network to be encrypted at all times.

Preference is given to infusion pumps and syringe pumps, but the examples are not limited to these. For example, it can also be a volumetric or peristaltic infusion pump.

The first infusion pump preferably sends digitally signed data or data with a digital signature to the second infusion pump and/or the second infusion pump sends encrypted data to the first infusion pump. This means that secure data transmission can be achieved on at least one side. This may already be sufficient for one infusion pump to send a command to the other, e.g., to take over a therapy. However, the data transmission can also take place reciprocally/in both directions or in the opposite direction. This means that, in addition or as an alternative, the second infusion pump can also send digitally signed data or data with a digital signature to the first infusion pump and/or the first infusion pump can send encrypted data to the first infusion pump. In other words, the infusion pump system can be set up for and/or carry out transmission of digitally signed data in both directions and/or transmission of encrypted data in both directions.

Preferably, the infusion pump system has at least two keys for decrypting and encrypting data and/or for generating and verifying digital signatures. In the case of transmission of digitally signed data and/or encrypted data in both directions, the infusion pump system can also have four such keys. However, the infusion pump system may also have more such keys, in particular if the infusion pump system has more than two infusion pumps or other devices.

In this disclosure, a key is to be understood as a digital key in the sense of cryptology/cryptography, and not, for example, a mechanical one.

The first infusion pump preferably has a first key and the second infusion pump a second key, preferably of the two keys as described above, wherein the first and second keys form a key pair. A key pair is preferably understood to mean two keys, where one encrypts a file and only the other/these two can then decrypt this file and/or one authenticates a file with a digital signature and the other validates it.

The first and second key can be identical/the same/a copy of each other, for example, a secret key for symmetric encryption, which can be used to encrypt and decrypt data. The first and second key can also be different. For example, the first and second keys can form a key pair with a private and a public key for an asymmetric crypto method/system for encryption-decryption and/or authentication-validation. Both infusion pumps can also have two keys each for a two-way asymmetric crypto method/system.

Preferably, the first infusion pump generates the first and second keys and the second infusion pump receives/has the second key from the first infusion pump. Alternatively or additionally, the second infusion pump can also generate a first and a second key and give the second key to the first infusion pump.

Preferably, the first key is a private key that only the infusion pump that creates it has, preferably the first infusion pump, and preferably the second key is a public key that can be sent to a plurality of infusion pumps, but at least to the/another infusion pump, preferably the second infusion pump. A private key is therefore not sent and is not intended for the public; a public key is sent/can be sent.

Preferably, the first, private key and the second, public key form a key pair in the sense of an asymmetric crypto method/system. This has the particular advantage that the transmission of the second key does not have to be done over a secure line/in a secure manner, since it is a public key. Thus, the second key can be transferred to enable secure data transmission afterwards, wherein the line does not have to be secure at this point in time/it is not necessary for the line to be secure in order to exchange keys.

Preferably, the first key is set up to provide data sent by its owner/producer, preferably the first infusion pump,

with a unique, individual, forgery-proof digital signature in order to identify who the data is or originates from. The second key is preferably set up to check a digital signature of data and to validate the digital signature of the first key or to decide, based on the digital signature, whether the data is or originates from the owner of the first key.

It is to be understood that the keys themselves do not need to fulfill these functions, but that the owner of the respective key can do so with the key/with its help.

The owner of the second key, preferably the second infusion pump, accepts data for which the second key or for which, based on the digital signature of the data, the second key decides that it originates from the owner of the first key, preferably the first infusion pump. The owner of the second key, preferably the second infusion pump, discards data for which the second key, or rather the second key based on the digital signature of the data, decides that it does not originate from the owner of the first key, preferably the first infusion pump.

Thus, the second infusion pump then discards, for example, non-validated commands or commands that do not originate from the validated first infusion pump. This ensures that manipulated data and/or data from an untrusted source does not deceive/manipulate the second infusion pump.

It should be understood that this applies to a data transmission in the infusion pump system or between infusion pumps/to an infusion pump from an external device, e.g., infusion pump, but not to an input to an infusion pump. In other words, the infusion pumps naturally accept, for example, manual or direct input from users such as doctors or nurses, and do not discard it. The same preferably also applies to authorized/validated devices, for example computers, input devices or further infusion pumps.

Discarding can include ignoring, deleting, not even saving, not even accepting, and not even opening the data.

It is to be understood that it can be decided on the basis of the digital signature whether the data originates from the owner of the first key, and this means that if the digital signature is different or missing, the data does not originate from the owner of the first key and/or the data has been modified.

The second key is preferably set up/able to encrypt data in such a way that it can only be decrypted by the first key. Preferably, only the first key is set up/is able to decrypt data that is/was encrypted by the second key. This means that intercepted data cannot be decrypted and manipulated.

It is to be understood that the keys themselves do not need to fulfill these functions, but that the owner of the respective key can do so with the key/with its help.

The infusion pumps are set up to generate and output pairing information. Furthermore, the infusion pumps are set up to send the pairing information to at least one other infusion pump and to receive it from one other infusion pump. The pairing information can be, for example, a numerical code, word, color code, or similar. The disclosure is not limited to this, however; it could also be an audio signal, for example. The pairing information is preferably generated by an infusion pump, preferably the first infusion pump, and sent to at least one/the other infusion pump, preferably the second infusion pump.

Preferably, at least one infusion pump or the infusion pumps are set up to register a confirmation for pairing, preferably a manual input by a user. In other words, at least one infusion pump, or preferably all infusion pumps, can receive and process a confirmation for pairing based on the pairing information, e.g., by matching the pairing information, in order to then carry out the pairing. Preferably, the pairing is not carried out without the confirmation. In other words, the pairing is preferably confirmed and thus authorized, preferably by a user. A particularly preferred exemplary embodiment is characterized in that the first infusion pump and the second infusion pump are set up to output, preferably display, pairing information, and at least one of the first infusion pump and the second infusion pump is set up, preferably both infusion pumps are set up, to register a confirmation of the pairing based on the pairing information. In other words, the confirmation for pairing can be done on both infusion pumps or, alternatively, only on one infusion pump.

The pairing information is information for pairing at least two infusion pumps together. A user, for example a doctor or nurse, can then pair the at least two infusion pumps by comparing the pairing information displayed by the at least two infusion pumps. If the pairing information matches, the user can confirm this on at least one of the infusion pumps or both infusion pumps, thereby authorizing the pairing so that the pairing is carried out. This means that secure data transmission is possible and has been checked and confirmed by a user. This increases security. It is to be understood that if the user authorizes/confirms the pairing, it is preferred that the pairing be carried out, and that the keys for this be kept or accepted and then used for secure data transmission. If the user does not authorize the pairing, for example because the pairing information does not match, the keys are preferably discarded/deleted.

The first and second infusion pump infusion pumps) are preferably set up to be paired and unpaired with each other. It is preferable for the infusion pumps to be unpaired after a patient's therapy has ended; they are particularly preferably set up to do this independently. The respective keys are preferably discarded when unpaired. For example, the first infusion pump can discard/delete its key and digitally sign a command to the second infusion pump to do the same with its key.

The first and second infusion pump infusion pumps) are preferably set up to create, send, receive, and/or evaluate/process a request for a pairing. Preferably, the slave sends a request to the master in a take-over mode. This means that the infusion pump preferably requests a pairing, which then receives a command, preferably to start or take over the therapy, from the other infusion pump.

The first and second infusion pump are preferably set up to accept or reject a request for pairing, preferably depending on whether they are available. Available infusion pumps are, for example, operational infusion pumps, those with suitable and/or sufficient drug provided and/or suitable infusion pumps that can be paired/are pairable/can form an infusion pump system, preferably as described above. Available for pairing can also be understood to mean that they are set up and/or ready to do so, for example, are not already running/active. A user can also preferentially select/decide which infusion pumps are paired and/or which send a request and/or which accept or possibly reject it.

Preferably, the infusion pumps are set up to make a request for pairing if they will/could no longer be available in the foreseeable future. This means, for example, that an infusion pump that has finished administering its drug, whose drug is running low or that has a fault/problem can/will request a pairing.

The infusion pumps are particularly set up to request a pairing when they are ready or available and/or when, for example, a drug is inserted into the infusion pump and/or, preferably via a user interface, a therapy is selected.

A request preferably contains information about a therapy to be carried out. For example, the request preferably contains which drug the infusion pump is showing/has been inserted and/or which therapy has been/is being selected. For example, it can also be included in a request as to which drug and which quantity of it an infusion pump should provide/show.

The infusion pumps can be set up to send a request when a user enters the appropriate information. The user can preferably select which infusion pump(s) the request is sent to and/or which pump(s) accept the request. The infusion pumps can be set up to reject or accept a request when a user enters the appropriate response.

The infusion pumps are set up to display their status, for example, whether they are running, paused, or available.

The infusion pump system may further comprise a controller that send encrypted and/or digitally signed data to and/or receive encrypted and/or digitally signed data from at least one of the first and second infusion pumps. The controller may be a device that is preferably operated by a user of the infusion pump system, such as a computer, an input device, and/or another infusion pump. The controller can also be present instead of the first or second infusion pump of the infusion pump system and/or have/take over their features/function(s).

The disclosure further relates to a method for secure data transmission for an infusion pump system, preferably as described above, having a first infusion pump and a second infusion pump, comprising the following steps, preferably in this order:
pairing the first and second infusion pump;
receiving data signed with a digital signature for at least one of the first and second infusion pump; and/or
receiving data encrypted with a cipher for at least one of the first and second infusion pumps.

Furthermore, the step of pairing the first and second infusion pumps can comprise the following, preferably as described above, and/or the following steps, preferably in this order:
generating a first key and a second key from the first infusion pump;
generating and outputting pairing information, preferably to a user of the infusion pump system, from the first infusion pump;
transmitting the pairing information and the second key to the second Infusion pump;
outputting the pairing information from the second infusion pump, preferably to the user of the infusion pump system;
checking the pairing information displayed by the first infusion pump and the second infusion pump, preferably by the user of the infusion pump system;
with identical pairing information, confirmation thereof, preferably by the user of the infusion pump system, and pairing of the first infusion pump and the second infusion pump for receiving data with a digital signature for at least one of the first and second infusion pumps and/or receiving encrypted data for at least one of the first and second infusion pumps.

It is to be understood that it is therefore preferred when, the pairing is performed, and the keys to do so are kept or accepted and then used for secure data transmission. If the user does not authorize the pairing or does not complete it, for example because the pairing information does not match, the keys are preferably discarded/deleted.

It is to be understood that the pairing of the first and second infusion pump may also include pairing more than two infusion pumps and/or with at least one controller, preferably as described above. Multiple copies of the second key can be made and sent to multiple infusion pumps and/or the at least one controller, so that there may be multiple infusion pumps that have the functions and features of the second infusion pump. The pairing information can also be generated and sent multiple times, or the pairing can be carried out multiple times with different partners in succession. An infusion pump can also be paired to several infusion pumps at the same time.

It should also be understood that two key pairs can also be exchanged in both directions, each with a first and second key, so that there is a step/steps with:
generating a first key and a second key from the second key pair. Infusion pump;
transmitting this second key to the first infusion pump.

In other words, both infusion pumps can create a key pair from a first and second key, and send the second key to another infusion pump.

It is to be understood that the step of pairing as described above, preferably as described above, itself comprises/may comprise steps which can be regarded as a method for pairing a first and a second infusion pump, preferably as described above. In other words, the step of pairing the first and second infusion pumps can also be a method for pairing a first and second infusion pump. This pairing step/method can be carried out and completed preferentially before a patient is connected to one of the infusion pumps, and is thus not a method for the surgical or therapeutic treatment of the human or animal body and not a diagnostic method carried out on the human or animal body.

Furthermore, it should be understood that the step/method of pairing can take place once initially before data transmission(s) and also several times or before each data transmission.

The method for secure data transmission for an infusion pump system and/or the step/method of pairing may include the following step:
requesting the second infusion pump from the first infusion pump for pairing and/or requesting the second infusion pump from a plurality of available infusion pumps for a first infusion pump for pairing and selecting the first infusion pump from the plurality of available infusion pumps.

Furthermore, the method for secure data transmission for an infusion pump system and/or the step/method of pairing may include the following step:
accepting the request from the second infusion pump to the first infusion pump for pairing(s) from the first infusion pump.

If accepted, the other/further steps of the method for secure data transmission for an infusion pump system and/or the step/method of pairing, as described above, can then be carried out.

It is to be understood that a request for a pairing is preferably made first between two infusion pumps, and if the request is accepted by the first infusion pump, the pairing is then carried out between the first and second infusion pump, thus enabling secure data transmission. Therefore, the pairing is preferably confirmed and authorized by a user, preferably with the pairing information, in order to ensure that a request, which could be intercepted and falsified due to the as-yet-unsecure data transmission, does not automatically lead to a pairing.

A plurality of infusion pumps can be requested to be paired to form an infusion pump system, preferably as described above. Available infusion pumps are, for example, infusion pumps that are ready for operation, provided with suitable drug and/or suitable infusion pumps that can be paired/are pairable/can form an infusion pump system, preferably as described above. Available for pairing can also be understood here as being set up and/or ready for it. It can also request the first infusion pump from the second infusion pump or a plurality of infusion pumps.

The method for secure data transmission for an infusion pump system can further comprise the following steps, preferably in this order:
sending data with a unique and individual digital signature, the first key created by the first infusion pump;
checking the digital signature of data with the second key from the second infusion pump and decision based on the digital signature as to whether the data originates from the first infusion pump;
acceptance of the data by the second infusion pump if the decision shows that the data originates from the first infusion pump; or
discarding the data by the second infusion pump if the decision shows that the data does not originate from the first infusion pump; and/or the following further steps:
encrypting data with the second key from the second key Infusion pump;
sending the encrypted data from the second infusion pump;
receiving the encrypted data from the first infusion pump;
decrypting the data with the first key from the first infusion pump

As described above, data encrypted in both directions or in different directions and/or data digitally signed in both directions can also be transmitted. Accordingly, the first and second infusion pump can also be exchanged and/or the second infusion pump can additionally exhibit/perform the features/functions/steps of the first infusion pump and the first infusion pump can additionally exhibit/perform the features/functions/steps of the first infusion pump.

The method for secure data transmission for an infusion pump system and/or the step/method of pairing may include the following step:
unpairing from the paired infusion pumps.

This step can also be performed even if no patient is connected to the infusion pumps. Preferably, the unpairing takes place when a patient's therapy has ended. The respective keys are preferably discarded when unpaired.

The disclosure further relates to a computer readable storage medium having functions that cause an infusion pump system, preferably as described above, to perform steps of a method of secure data transmission for an infusion pump system and/or a method of pairing, preferably as described above.

### BRIEF DESCRIPTION OF THE DRAWINGS

The disclosure is explained in more detail below, with reference to preferred embodiments and the accompanying figures.

FIG. 1 shows a request from an infusion pump for a pairing for secure data transmission in accordance with the present disclosure;

FIG. 2 shows two infusion pumps being paired for secure data transmission in accordance with the present disclosure;

FIG. 3 shows transmission of digitally signed data between two infusion pumps according to the present disclosure;

FIG. 4 shows encrypted data being transmitted between two infusion pumps in accordance with the present disclosure.

FIG. 5 shows a two-way transmission of digitally signed data between two infusion pumps according to the present disclosure.

FIG. 6 illustrates a two-way transmission of encrypted data between two infusion pumps according to the present disclosure.

FIG. 7 shows an infusion pump system with a controller and secure data transmission according to the present disclosure;

FIG. 8 shows further secure data transmission to FIG. 7;

FIG. 9 shows a request from an infusion pump for a pairing for secure data transmission according to the present disclosure;

FIG. 10 corresponds to FIG. 2;

FIG. 11 corresponds to FIG. 3;

FIG. 12 shows a request from an infusion pump in FIG. 9 to another infusion pump for a pairing for a secure data transmission according to the present disclosure;

FIG. 13 shows a computer-readable storage medium.

The figures are schematic and serve only to aid understanding of the disclosure. The features of the various embodiments can be replaced with each other.

### DETAILED DESCRIPTION

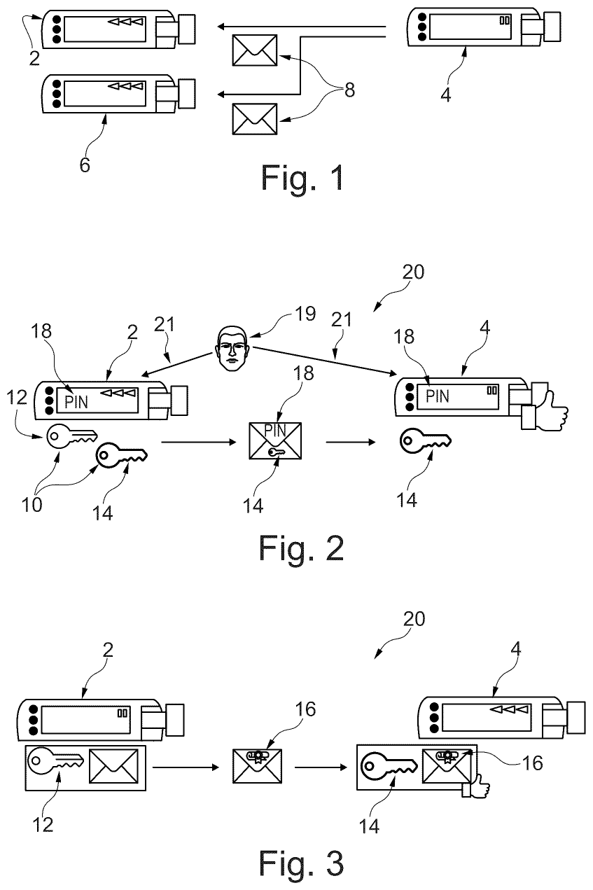

In FIG. 1, a first infusion pump 2 is requested by a second infusion pump 4 for a pairing for secure data transmission. The second infusion pump 4 also requests a third infusion pump 6. The second infusion pump 4 asks the other infusion pumps available, in this case the other two. To do this, it sends a request 8 to the available infusion pumps. Here, the first infusion pump 2 accepts the request 8 from the second infusion pump 4.

FIG. 2 shows the first infusion pump 2 and the second infusion pump 4 from FIG. 1. The first infusion pump 2 generates a key pair 10 with a first key 12 and a second key 14. The first key 12 here is a private key and is set up to provide data sent by the first infusion pump 2 with a unique and individual, forgery-proof digital signature 16 to identify that it originates from the first infusion pump 2. The second key 14 is a public key set up to check a digital signature 16 of data and to decide whether the digital signature 16 is from the first key 12 and the data thus originates from the first infusion pump 2 or whether it has been modified or sent from somewhere else. The key pair 10 is therefore asymmetric here, or for an asymmetric crypto method/system. The first infusion pump 2 generates a pairing information 18 “PIN” and outputs it for a user 19, e.g., a doctor or a nurse. Then, or while doing so, the first infusion pump 2 sends the pairing information 18 and the second key 14 to the second infusion pump 4. The second infusion pump 4 now has the second key 14 and the pairing information 18, and outputs/displays the pairing information 18 for the user 19. The user 19 compares the pairing information 18 displayed by the infusion pumps 2, 4. Since they are identical/match, he performs a confirmation 21 and thus authorizes the pairing, whereupon the first infusion pump 2 and the second infusion pump 4 are paired with each other, that is, they can/do transmit digitally signed data and/or encrypted data with the present keys securely in order to perform a therapy of a patient together simultaneously or in succession. The first and second infusion pump 2, 4 are therefore part of/form an infusion pump system 20.

FIG. 3 shows the infusion pump system 20 from FIG. 2 and illustrates the transmission of digitally signed data for secure data transmission. The first infusion pump 2 signs the data with the first key 12 and then sends it to the second infusion pump 4. The controller checks the digital signature 16 of the data with the second key 14 to see whether this digital signature 16 is from the first key 12 and thus the data is unambiguous and unaltered and originates from the first infusion pump 2. This is the case here and the data is accepted by the second infusion pump 4. Otherwise, they would be rejected.

FIGS. 1, 2, and 3 together show a take-over mode with secure data transmission according to the present embodiment. Initially, the first infusion pump 2 in FIG. 1 performs a specific therapy of a patient with a specific drug. The second infusion pump 4 is prepared to carry out a therapy, in this case the therapy of the first infusion pump 2 with the same drug, or to take it over. The second infusion pump 4 is initially not active. The second infusion pump 4 sends the request 8, in which it communicates parameters, i.e., for which therapy it is prepared. In this case, the first infusion pump 2 carries out the corresponding/this/the same therapy, the third infusion pump 6 carries out a different therapy. This means that the first infusion pump 2 recognizes from request 8 that the second infusion pump 4 is prepared/suitable to take over the implementation of the therapy. The first infusion pump 2 and the second infusion pump 4 are then pairing as described above and shown in FIG. 2. Then, as shown and described in FIG. 3, the first infusion pump 2 sends digitally signed data to the second infusion pump 4. In this case, it is a command/instruction to start, i.e., to take over the implementation of the therapy. The second infusion pump 4 now takes over the therapy and administers its drug, i.e., it is now active and the first infusion pump 2 is paused/no longer active. This ensures that therapy is/can be maintained, that there are no interruptions and that transitions between infusion pumps are seamless, with secure and reliable data exchange between the pumps. The second infusion pump 4, which is making the request 8 here, is a slave that receives digitally signed command(s) from the master, in this case the instruction to start from the first infusion pump 2. For example, the nurse sends the instruction to start when he has administered his drug. The slave then takes over the therapy from the master.

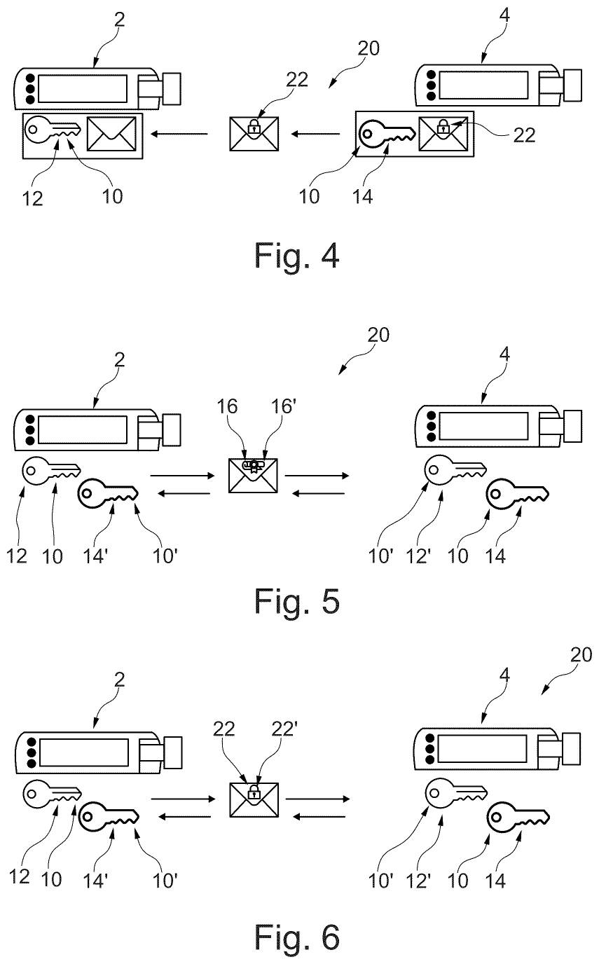

FIG. 4 shows the transmission of encrypted data. FIG. 4 shows the infusion pump system 20 from FIG. 3, however, the second key 14 is additionally or alternatively set up to encrypt data so that it can only be decrypted with the first key 12. The first key 12 is set up to decrypt data that was encrypted with the second key 14. The key pair 10 is therefore also an asymmetric key pair, or rather for an asymmetric crypto method/system, wherein the first key 12 is also the private key and the second key 14 is also the public key. The second infusion pump 4 encrypts data that it sends to the first infusion pump 2 with an encryption 22, so that the data can only be decrypted by the first key 12 or the first infusion pump 2 with the first key 12. The first infusion pump 2 then decrypts the encryption 22 and can read and process the data.

FIG. 5 shows the infusion pump system 20 from FIG. 3, however, the second infusion pump 4 also generates a second key pair 10′ with a first digital key 12′ and a second digital key 14′. The second infusion pump 4 initially transmits the second key 14′ to the first infusion pump 2 when they are paired, so that the first infusion pump 2 now has the second key 14′ of the second key pair 10′. The first 12′ key is a private key and is set up to provide data sent from the second infusion pump 4 with a unique and individual, digital signature 16′ to identify that it originates from the second infusion pump 4. The second key 14′ is a public key set up to check a digital signature 16′ of data and determine whether the digital signature 16′ is from the first key 12′ and the data thus originated from the second infusion pump 4 or whether it has been modified or sent from somewhere else. The key pair 10′ is therefore asymmetrical here, or rather for an asymmetrical crypto method/system. In this way, both infusion pumps 2 and 4 can alternately provide and send data with digital signatures 16 and 16′ with the respective first key 12 and 12′, and the respective other infusion pump 4 and 2 can check the respective signature with the respective second key 14 and 14′ and thus ensure that the data really originates from the other infusion pump 4 and 2. This is done as illustrated in FIG. 5.

FIG. 6 shows the infusion pump system 20 from FIG. 5. However, the second keys 14, 14′ are set up in addition or as an alternative to provide data with encryption 22 or 22′ in such a way that it can only be decrypted by the corresponding first key 12 or 12′ of the respective key pair 10 or 10′. the respective first keys 12,12′ are set up to decrypt data that has been encrypted by the respective/corresponding second key 14, 14′. Thus, the first infusion pump 2 and the second infusion pump 4 can transmit securely encrypted data in both directions and do so, wherein the keys encrypt and decrypt accordingly. The key pairs 10 and 10′ are therefore asymmetrical, or rather, for an asymmetrical crypto method/system.

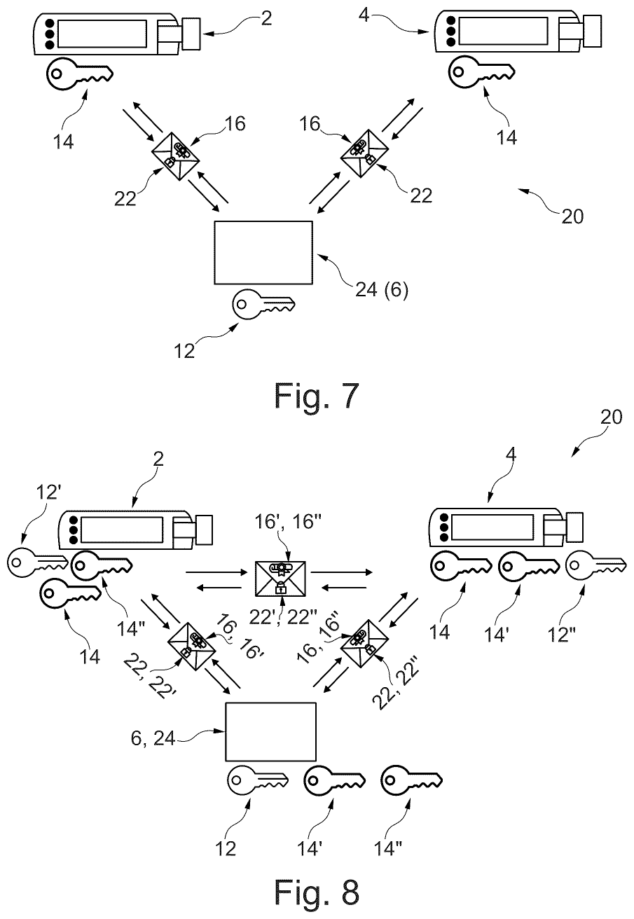

FIG. 7 shows an infusion pump system 20 with two infusion pumps 2, 4 and a controller 24. The controller 24 can have the function and/or features of the first and/or second infusion pumps 2, 4 from one of the previous figures. The controller 24 can also be a third infusion pump 6 itself. In this case, the controller 24 is paired with the two infusion pumps 2, 4. It has a first, private key 12 itself, which has/may have the characteristics from the/one of the previous figures, and the infusion pumps 2, 4 each have an associated second key 14, which has/may have the characteristics from the previous figures. Thus, the controller 24 can send digitally signed/with a unique, tamper-proof digital signature 16 data to one and/or both infusion pumps 2, 4 to securely send instructions to them, for example, and they can check the digital signature 16, as previously described. This is also how it is done. Additionally or alternatively, the infusion pump(s) 2, 4 can securely encrypt data/data with an encryption 22 and only the controller 24 can decrypt it with the first key 12, as described above. This is also done in the same way.

FIG. 8 shows a two-way secure data transmission in an infusion pump system 20 with two infusion pumps 2, 4 and a controller 24, wherein the controller 24 can also be a third infusion pump 6. Data encrypted with an encryption 22 or 22′ or 22″ and/or data signed with a digital signature 16 or 16′ or 16″ can securely sent between all devices of the infusion pump system 20 in both directions. Each of the devices of the infusion pump system 20 has a private first key 12 or 12′ or 12″ and two public second keys 14′ and 14″ or 14 and 14″ or 14 and 14′ associated with the first keys of the other devices.

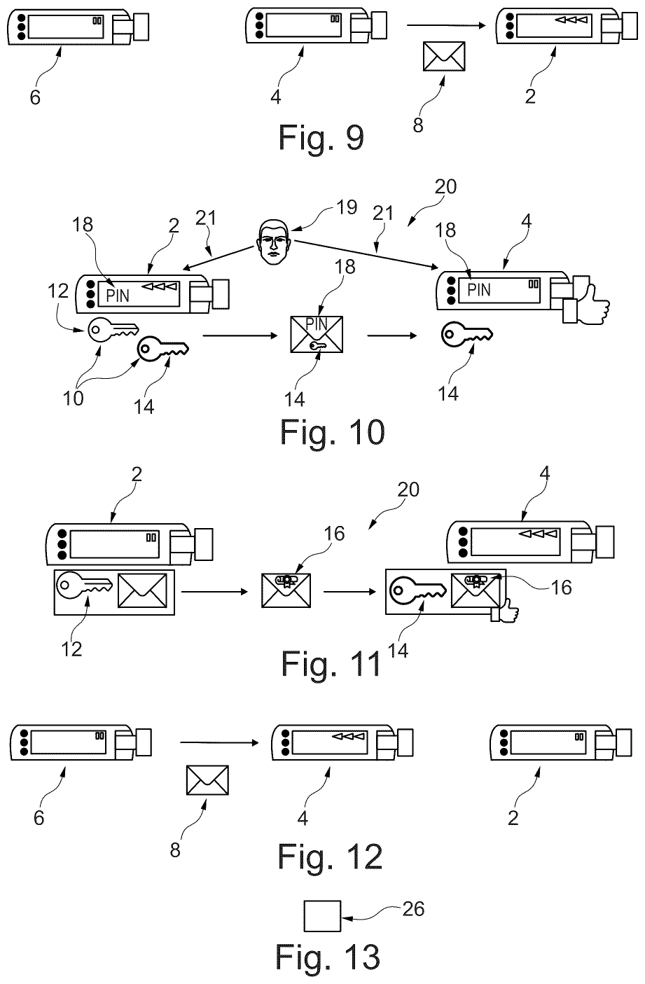

FIG. 9 corresponds to FIG. 1, wherein the third infusion pump 6 is not exemplarily executing an therapy, but is inactive. The second infusion pump 4 also sends the request 8 to the third infusion pump 6, but it rejects it.

FIG. 10 corresponds to FIG. 2 and shows the pairing of the first infusion pump 2 with the second infusion pump 4.

FIG. 11 corresponds to FIG. 3 and shows that the first infusion pump 2 digitally signs the command sent to the second infusion pump 4 to start and take over the therapy.

FIG. 12 shows the request from the third infusion pump 6 to the second infusion pump 4 to establish a pairing. The third infusion pump 6 is now available and set up/ready to take over the therapy from the second infusion pump 4. For example, the user 19 has just previously filled or selected the drug or selected a therapy and thus the third infusion pump 6 now makes the request 8.

FIGS. 9 through 12 thus illustrate a takeover mode wherein a total of three infusion pumps perform a therapy together in succession. First, the first infusion pump 2 in FIG. 9 does this. The second infusion pump 4 requests the first infusion pump 2 for a pairing. The first infusion pump 2 and the second infusion pump 4 are paired in FIG. 10. The first infusion pump 2 as the master then digitally signs and thus securely transmits the instruction to start to the second infusion pump 4, the slave. In this case, the second infusion pump 4 will administer the therapy. In FIG. 12, the third infusion pump 6 is ready to continue the therapy and requests the second infusion pump 4 for a pairing. The second infusion pump 4 and the third infusion pump 6 are then paired and the second infusion pump 4, as the master, instructs the third infusion pump 6, as the slave, to start and thus take over the therapy. Thus, the therapy is initially carried out by the first infusion pump 2, then the second infusion pump 4 and then the third infusion pump 6. The master status is thus also transferred from the first infusion pump 2 to the second infusion pump 4 and then to the third infusion pump 6, and the salve status is transferred from the second infusion pump 4 to the third infusion pump 6. In this process, the slave always becomes the master. This means that any number of infusion pumps can be connected in series/switched together, perform a therapy together, and pass the execution of a therapy on to the next infusion pump.

FIG. 13 shows a computer-readable storage medium 26 having functions that cause an infusion pump system 20, preferably as described above, to execute steps of a method for secure data transmission for an infusion pump system 20, preferably as described above, and/or a method for pairing, preferably as described above.

### LIST OF REFERENCES

2 first infusion pumps
4 second infusion pump
6 third infusion pump
8 request
10, 10′, 10″ key pair
12, 12′, 12″ first key
14, 14′, 14″ second key
16, 16′, 16″ digital signature
18 pairing information
19 user
20 infusion pump system
21 confirmation
22, 22′, 22″ encryption
24 controller
26 computer-readable storage medium

1. An infusion pump system comprising:
a first infusion pump; and
a second infusion pump,
the first infusion pump and the second infusion pump being paired or pairable to each other for secure data transmission,
the infusion pump system being configured so that:
at least one of the first infusion pump and the second infusion pump receives data signed with a digital signature, and/or
at least one of the first infusion pump and the second infusion pump receives encrypted data using an encryption.

2. The infusion pump system according to claim 1, wherein the first infusion pump and/or the second infusion pump is configured to output a pairing information.

3. The infusion pump system according to claim 2, wherein the first infusion pump and/or the second infusion pump is configured to display the pairing information.

4. The infusion pump system according to claim 2, wherein the first infusion pump and/or the second infusion pump is further configured to register a confirmation for a pairing based on the pairing information.

5. The infusion pump system according to claim 1, wherein the infusion pump system is configured so that:
the first infusion pump sends data with a digital signature to the second infusion pump, and/or
the second infusion pump sends encrypted data to the first infusion pump.

6. The infusion pump system according to claim 1, further comprising at least two keys for decrypting and encrypting data and/or for generating and checking digital signatures.

7. The infusion pump system according to claim 6, wherein:
the at least two keys comprises a first key for the first infusion pump and a second key for the second infusion pump, for pairing for secure data transmission.

8. The infusion pump system according to claim 7, wherein the first infusion pump generates the first key and the second key, and the second infusion pump has the second key from the first infusion pump.

9. The infusion pump system according to claim 7, wherein:
the first key is a private key that only the first infusion pump has,
the second key is a public key that is sent from the first infusion pump to the second infusion pump, and
the second key is configured for sending to one or more other infusion pumps.

10. The infusion pump system according to claim 7, wherein:
the first key is configured to provide data sent from the first infusion pump with a unique and individual digital signature to identify that said data originates from the first infusion pump, and
the second key is configured to check a digital signature of data sent to the second infusion pump and to decide whether the digital signature is from the first key and the data thus originates from the first infusion pump.

11. The infusion pump system according to claim 10, wherein:
the second infusion pump accepts data that the second key decides, based on the digital signature, originates from the first infusion pump, and
data which the second key decides, based on the digital signature, does not originate from the first infusion pump, is discarded.

12. The infusion pump system according to claim 7, wherein:
the second key is configured to encrypt data in such a way that said data is only decryptable by the first key, and
only the first key is configured to decrypt data encrypted by the second key.

13. The infusion pump system according to claim 1, further comprising a controller configured to at least one of:
send encrypted and/or digitally signed data to at least one of the first infusion pump and the second infusion pump, and
receive encrypted and/or digitally signed data from at least one of the first infusion pump and the second infusion pump.

14. A method for secure data transmission for an infusion pump system having a first infusion pump and a second infusion pump, the method comprising the steps of:
pairing the first infusion pump and the second infusion pump; and
receiving at least one of:
data signed with a digital signature for at least one of the first infusion pump and the second infusion pump, and
data encrypted with an encryption for at least one of the first infusion pump and the second infusion pump.

15. The method according to claim 14, wherein the step of pairing the first infusion pump and the second infusion pump comprises:
generating a first key and a second key from the first infusion pump;
generating and outputting a pairing information from the first infusion pump;
transmitting the pairing information and the second key to the second infusion pump;
outputting the pairing information from the second infusion pump;
checking the pairing information displayed by the first infusion pump and the second infusion pump;
confirming that the pairing information displayed by the first infusion pump is identical to the pairing information displayed by the second infusion pump; and
pairing the first infusion pump and the second infusion pump for receiving data with a digital signature for at least one of the first infusion pump and the second infusion pump and/or receiving encrypted data for at least one of the first infusion pump and the second infusion pump.

16. The method according to claim 15, further comprising the steps of:
sending data with a digital signature created by the first key from the first infusion pump;
checking the digital signature with the second key of the second infusion pump;
determining, based on the digital signature, whether the data originates from the first infusion pump; and
one of:
accepting the data by the second infusion pump when the data originates from the first infusion pump; or
discarding the data by the second infusion pump when the data does not originate from the first infusion pump.

17. The method according to claim 15, further comprising the steps of:
encrypting data with the second key from the second infusion pump;
sending the data that is encrypted from the second infusion pump;
receiving the data that is encrypted from the first infusion pump; and
decrypting the data that is encrypted with the first key from the first infusion pump.

18. The method according to claim 14, further comprising the step of:
requesting the second infusion pump from the first infusion pump for pairing; and/or
requesting the second infusion pump from a plurality of available infusion pumps for the first infusion pump for pairing and selecting the first infusion pump from the plurality of available infusion pumps.

19. A computer-readable storage medium having functions that cause an infusion pump system to perform the method according to claim 14.
